(19) United States
(12) Patent Application Publication
(10) Pub. No.: US 2010/0330133 A1
(43) Pub. Date: Dec. 30, 2010

(54) METHOD AND COMPOSITION FOR IMPROVING SKIN COMPLEXION

(75) Inventor: RICHARD A. PRESTI, AIRMONT, NY (US)

Correspondence Address: LADAS & PARRY LLP, 26 WEST 61ST STREET, NEW YORK, NY, 10023, US

(73) Assignee: BEILIS DEVELOPMENT INC.

(21) Appl. No.: 12/491,292

(22) Filed: Jun. 25, 2009

(51) Int. Cl. A61K 8/11; A61K 8/73; A61Q 19/08

(52) U.S. Cl. 424/401; 514/54

(57) ABSTRACT

Extracts of at least one, and preferably a mixture of, encapsulated mixed plant carbohydrates (polysaccharides) are applied topically to the skin to mitigate the appearance of wrinkled skin and to improve the overall complexion of the skin. The mixed plant extracts are preferentially applied in a dehydrated state, with a preferred source for the polysaccharides being cacti. The application vehicle is preferentially an anhydrous product, which allows slight penetration of smaller fractions of the dehydrated mixed cactus carbohydrate into the stratum corneum. Upon entering the stratum corneum the cactus carbohydrates begin to hydrate and swell resulting in a plumping effect, with the overall appearance of lines and wrinkles diminishing.

### FIELD OF THE INVENTION

The present invention relates to the topical application of encapsulated mixed plant carbohydrates and, more particularly, to a topical application of encapsulated mixed plant carbohydrates for the mitigation of wrinkles and improved skin complexion.

### BACKGROUND OF THE INVENTION

The skin is the body's largest organ, made up of multiple layers of epithelial tissues that protect the underlying muscles and organs. As the interface with the environment, the skin plays an important role in myriad activities including protection from the elements, maintaining body temperature, sensations, control of evaporation, synthesis of vitamins B and D, excretion of waste products, and exclusion of pathogens. Being the first line of defense, the skin is constantly being abused by environmental assaults such as the sun, wind, smog, pollution and temperature and humidity extremes while at the same time it is subjected to self-imposed damages such as soaps, solvents and hot water, all of which contribute to a decline in the skin's ability to function efficiently. These assaults result in the loss of skin moisture, free radical generation, and a build-up of xenobiotic substances, any of which can lead to skin with reduced barrier function, degradation in collagen and elastin, and accelerated skin aging. This skin aging manifests itself as wrinkled, dull, and/or sagging skin.

To date, the most effective treatments to mitigate the appearance of wrinkled and sagging skin are injections of Botox or hyaluronic acid based line fillers.

The shortcomings of existing treatments are few but important. First, treatments with Botox or line fillers are expensive and require one or several trips to a dermatologist. These treatments can be painful and cause redness and swelling for several days post-treatment. Finally, these treatments are transient, lasting at most nine months before follow-up visits to the dermatologist are required for additional treatments.

It is therefore an object of this invention to provide a cosmetic formulation and method of application for the mitigation of wrinkles and overall improvement of skin complexion.

### SUMMARY OF THE INVENTION

Purified polysaccharides from a plant source are able to associate with and bind a large amount of water. Upon solvation these polysaccharides can swell in a fashion similar to hyaluronic acid.

It is therefore an object of this invention to provide a system to deliver purified plant polysaccharides into the stratum corneum or upper epidermis of the skin to provide mitigation of wrinkles as well as to improve the overall complexion of the skin.

It is another object of this invention to provide a description of a delivery vehicle to most effectively deliver the plant polysaccharide to and into the skin.

It is a further object of the invention to identify an encapsulation material to protect the plant polysaccharide from premature hydration and enzymatic breakdown.

Finally, it is an object of this invention to identify a preferred plant source for the polysaccharides.

In accordance with the present invention, extracts of mixed plant polysaccharides (carbohydrates) are encapsulated and applied topically to the skin to mitigate the appearance of wrinkled skin and to improve skin complexion. Mixed plant polysaccharides have a tremendous affinity for water and swell when hydrated, allowing them to function in a similar fashion to hyaluronic acid. The mixed plant extracts are preferentially applied in a dehydrated state. Preferably, the plant polysaccharides are coated either covalently or ionically with a cosmetically acceptable coating agent, such as dextrin, dextran, cyclodextrin, a phospholipid and/or cholesterol, fatty alcohols or proteins. The application vehicle is preferentially an anhydrous product, which will allow slight penetration of smaller fractions of dehydrated mixed plant carbohydrates into the stratum corneum or upper epidermis. More preferably the vehicle further includes no free water. Upon entering the stratum corneum or upper epidermis the plant carbohydrates hydrate and swell, resulting in a plumping effect, with the overall appearance of lines and fine and coarse wrinkles diminishing, and the overall complexion of the skin improving. Polysaccharides from a single plant species or a further mixture of polysaccharides from a combination of plant species are within the contemplation of the invention. In addition, it is contemplated that various mixes of differing plant polysaccharides as extracted from plant matter can be compounded for use in the invention. At the present stage of development the efficacy of the invention is not believed to be based on a particular combination of polysaccharides, although as expressed herein certain plant families and species may be preferred.

### DESCRIPTION OF A PREFERRED EMBODIMENT

Cacti (family Cactaceae) are a distinct and unusual class of plants that have adapted to thrive in very hot and arid environments. These adaptations are evident in the typically very small leaves to reduce water loss, a waxy coating and the overall shape of the cactus itself, both of which serve to reduce water loss, and a high content of slats in the root system of the plant which enables a very quick uptake of water from the ground. Additionally, cacti have a high content of polysaccharides, which enables the plants to bind and hold onto large amounts of water. These polysaccharides have been shown to have the ability to bind large amounts of water through hydrogen bonding. Thus, cacti are a preferred source for the polysaccharides intended for use in the present invention.

Due to the size and charge of the cactus polysaccharides, penetration into the skin without a suitable protective coating or an improper delivery vehicle is not possible. In order to ensure adequate delivery of the cactus polysaccharides into the stratum corneum, a protective coating of the polysaccharides is necessary, as well as a specific anhydrous delivery vehicle containing appropriate penetration enhancers. Preferably, the polysaccharides have a particle size of less than 10,000 nm, and in the coated form have a size of between 500 and 5,000 nm.

Accordingly, mixed cactus polysaccharides are preferentially coated either covalently or ionically to hinder immediate hydration of the cactus material as well as to limit any enzymatic degradation of the polysaccharides. This cosmetically acceptable coating can be, but is not limited to, dextrin, dextran, cyclodextrin, phospholipids and/or cholesterol, fatty alcohols, protein or methacrylate based polymers. The methods of coating are known in the art. Mixed polysaccharides are commonly available, and can be obtained, for example, from Independent Chemical Corp. of Glendale N.Y. 11385.

These coated cactus particles are preferably delivered topically to the skin via an anhydrous vehicle. The vehicle can consist of, but is not limited to, silicones or oils. Examples of silicone materials for the anhydrous vehicle include silicone elastomers, cyclomethicone, high and low viscosity dimethicone, siloxanes, and silanols. Examples of oil materials for the anhydrous vehicle include mineral oil, sunflower oil, sesame oil, soybean oil, rapeseed oil and tea tree oil. The anhydrous vehicle can also be composed of esters or aliphatic hydrocarbons.

Penetration of the mixed cactus polysaccharides can preferably be enhanced via cosmetically acceptable penetration enhancers in the delivery vehicle. Examples of penetration enhancers include, but are not limited to, dimethyl isosorbide, laurocapram, proplylene glycol, pentylene glycol, dimethyl sulfoxide, oleic acid, ethoxydiglycol, MSM, ethanol or the like as recognized by those skilled in the art. The encapsulated polysaccharides may be present at a rate of between 0.10 and 20.00 percent by total composition weight.

The combination of the encapsulation and the penetration enhancers allows penetration of the cactus material deep into the stratum corneum for maximum skin plumping effect.

Additional cosmetic active materials can be included in the vehicle. Examples of cosmetic active materials include, but are not limited to, emollients, peptides, plant and yeast extracts as well as synthetically derived materials.

In a most preferred embodiment, mixed cactus polysaccharides obtained from Ferocactus Wislizenii are coated with dextran and delivered to the skin with a silicone elastomer based vehicle containing penetration enhancers.

Since other modifications and changes varied to fit particular operating requirements and environments will be apparent to those skilled in the art, the invention is not considered limited to the example chosen for purposes of disclosure, and covers all changes and modifications which do not constitute departures from the true spirit and scope of this invention.

What is claimed is:

1. A method to improve the appearance of fine lines and fine or coarse wrinkles and to improve the overall complexion of the skin, comprising the topical application of an effective amount of at least one encapsulated plant polysaccharide in an anhydrous vehicle.

2. The method of claim 1 where the at least one polysaccharide is mixed cacti polysaccharides at a concentration of the mixed cactus polysaccharides of between 0.10% and 20.00% by weight.

3. The method of claim 2 wherein the cacti polysaccharides are encapsulated with a material chosen to delay hydration and hinder enzymatic breakdown of the polysaccharides.

4. The method of claim 3 wherein the polysaccharides are encapsulated by a material from the group comprising dextrin, dextran, cyclodextrin, phospholipids, cholesterol, fatty alcohols and proteins.

5. The method of claim 2 wherein the vehicle contains no free water.

6. The method of claim 1 wherein the size of the encapsulated plant polysaccharides is between 500 and 5,000 nanometers.

7. The method of claim 1 wherein the topical application includes the simultaneous application of a penetration enhancer for the at least one polysaccharide.

8. The method of claim 7 wherein the penetration enhancer is chosen from the group comprising dimethyl isosorbide, laurocapram, propylene glycol, pentylene glycol, dimethyl sulfoxide, oleic acid, ethanol and ethoxydiglycol.

9. The method of claim 2 wherein the mixed polysaccharides are obtained from Ferocactus Wislizenii.

10. A composition for improving the appearance of fine lines and fine or coarse wrinkles and to improve the overall complexion of the skin, comprising mixed encapsulated plant polysaccharides in an anhydrous vehicle.

11. The composition of 10 where the mixed polysaccharides are cacti polysaccharides at a concentration of between 0.10% and 20.00% of total composition weight.

12. The composition of claim 10 wherein the cacti polysaccharides are encapsulated with a material chosen to delay hydration and hinder enzymatic breakdown of the polysaccharides.

13. The composition of claim 11 wherein the polysaccharides are encapsulated with a material from the group comprising dextrin, dextran, cyclodextrin, phospholipids, cholesterol, fatty alcohols and proteins.

14. The composition of claim 10 wherein the size of the encapsulated plant polysaccharides is between 500 and 5,000 nanometers.

15. The composition of claim 10 wherein the vehicle includes at least one penetration enhancer for the polysaccharides.

16. The composition of claim 15 wherein the at least one penetration enhancer is chosen from the group comprising dimethyl isosorbide, laurocapram, propylene glycol, pentylene glycol, dimethyl sulfoxide, oleic acid, ethanol and ethoxydiglycol.

17. The composition of claim 16 wherein the concentration of penetration enhancers is between 0.10% and 20.00% by weight.

18. The composition of claim 10 wherein the polysaccharides are obtained from Ferocactus Wislizenii.
